(19) United States
(12) Patent Application Publication
(10) Pub. No.: US 2015/0216444 A1
(43) Pub. Date: Aug. 6, 2015

(54) PATIENT BED WITH MAGNETIC RESONANCE RADIO FREQUENCY ANTENNA, PARTICULARLY FOR USE IN A MAGNETIC RESONANCE IMAGING GUIDED THERAPY SYSTEM

(71) Applicant: KONINKLIJKE PHILIPS N.V., EINDHOVEN (NL)

(72) Inventor: Falk Uhlemann, Hamburg (DE)

(21) Appl. No.: 14/420,762

(22) PCT Filed: Jul. 25, 2013
(86) PCT No.: PCT/EP2013/065720

§ 371 Date: Feb. 10, 2015

Related U.S. Application Data
(60) Provisional application No. 61/682,904, filed on Aug. 14, 2012.

(30) Foreign Application Priority Data
Aug. 14, 2012 (EP) 12180400.9

(51) Int. Cl. A61B 5/055; G01R 33/30; A61G 7/075; G01R 33/34

(52) U.S. Cl. CPC A61B 5/0555; G01R 33/34; G01R 33/307; A61G 7/0755

(57) ABSTRACT

A patient bed, particularly for use in a magnetic resonance (MR) imaging-guided therapy system employing at least one out of ionizing radiation and ultrasound energy for therapy purposes, having an abdominal support portion (14) for supporting an abdominal region (18) of a subject (12) during magnetic resonance-guided therapy, comprising at least one magnetic resonance (MR) radio frequency (RF) antenna device (48) arranged at a top side (26) of the patient bed in a patient bed center region (30), with at least one MR RF antenna (50) that is enclosed in a housing (52) having two side surfaces (54) opposing each other, wherein, in at least one state of operation, each side surface (54) of the MR RF antenna device (48) is provided to be proximal to an inner side of each of the subject's legs (22), and wherein, in the at least one state of operation, the MR RF antenna device (48) is provided to be proximal to a subject's perineum (20); an MR radio frequency (RF) antenna device (48) therefor; and a therapy system employing at least one out of ionizing radiation and ultrasound energy for therapy purposes that is guided by an MR imaging device with a patient bed having at least one MR radio frequency (RF) antenna device (48).

### FIELD OF THE INVENTION

The invention pertains to a patient bed for supporting an abdominal region of a subject, in particular during magnetic resonance (MR)-guided radiotherapy, and an MR radio frequency (RF) antenna device therefor, and a radiotherapy system guided by an MR imaging device with a patient bed having an MR radio frequency (RF) antenna device.

### BACKGROUND OF THE INVENTION

In the art of therapy systems guided by magnetic resonance imaging devices, integrated magnetic resonance-therapy (MR-T) systems are known that allow for real-time MR-monitoring of a patient during therapy. The therapy systems may employ at least one out of ionizing radiation and ultrasound energy for therapy purposes.

One of the most likely areas of application for MR-T systems is the treatment of abdominal organs like prostate gland, cervix, kidney, and bladder, as these exhibit significant movements or deformations in between and during radiotherapy sessions.

Since tracking of the organs and lesions is the primary objective of the integrated MR imaging, a clear imaging of organ or lesion boundaries is essential. Maximum diagnostic quality, on the other hand, is not required as the respective imaging is done in a preliminary step before therapy planning.

For diagnostic purposes demanding highest image quality, these organs are imaged employing special local MR RF antennae (coils), for instance endorectal antennae. Because the local MR RF antennae can be placed in proximity of the target region, for instance a prostate gland, significantly better signal-to-noise ratios are achievable that may be about ten times larger than with a body coil. From a workflow and patient comfort perspective, however, they are not practical in fractionated therapy treatments that require frequent placement and disinfection for several treatments per week over several weeks, while bowel syndromes are often occurring due to concurrent chemotherapy.

MR RF antennae designed as surface coils are known to allow for higher signal-to-noise ratios by local imaging without any need for an intracavitary application, but are difficult to be positioned reproducibly at an exact same location, which can lead to problematic absorption compensation issues and hinder an efficient work flow.

For MR-T devices, MR imaging employing the MRI system body coil is possible but not sufficient for organ or lesion delineation (e.g. in case of the prostate gland) due to the lower signal-to-noise ratio.

A high patient throughput of therapy treatments demands an efficient workflow (treatment session interval duration of 10 to 15 minutes). Maximum patient comfort is desirable because the fractionation of the treatment dose splits the treatment into 20 or more radiation sessions.

In view of radiotherapy treatment session interval durations of 10 to 15 minutes and a split of the treatment into 20 or more radiation sessions, it is desirable to provide means for a magnetic resonance (MR)-guided radiotherapy with an improved patient throughput of therapy treatments and an improved patient comfort.

### SUMMARY OF THE INVENTION

It is therefore an object of the invention to provide a patient bed having a supporting portion for supporting an abdominal region of a subject, usually a patient, during magnetic resonance (MR)-guided therapy employing at least one out of ionizing radiation and ultrasound energy for therapy purposes and having a lying direction intended for the subject to lie along.

The patient bed comprises at least one magnetic resonance (MR) radio frequency (RF) antenna device with at least one MR RF antenna that is enclosed in a housing having two side surfaces opposing each other, wherein the MR RF antenna device is arranged at a top side of the patient bed in a patient bed center region. The MR RF antenna may be of a coil-type design or of any other design that appears to be appropriate to the one skilled in the art. The housing may be made from any non-magnetic material fulfilling the requirements of sanitizing that apply in hospitals or medical centers, while providing sufficient mechanical strength.

The MR RF antenna device further comprises a radiation detecting device that is enclosed in the housing. The radiation detecting device may allow measuring an incident therapy radiation at a known location, which provides real-time data about the radiation at the known location for quality assurance, verification and documentation purposes. Preferably, the radiation detecting device is arranged at a position within the portion of the housing that encompasses the headmost one third of the housing relative to the lying direction of the patient bed. By that, the radiation detecting device is located close to but outside regions of radiation exposure within the subject, and may serve quality assurance purposes by measuring a stray dose at the known location. It may further allow to compare the measured radiation dose with planning data in real-time. In case of a detected deviation from the planning data, a radiation treatment can be quickly corrected or interrupted.

Further, the patient bed comprises two leg support portions for supporting the subject's legs from below, wherein, in at least one state of operation, each side surface of the MR RF antenna device is provided to be proximal to an inner side of each of the subject's legs; and wherein, in the at least one state of operation, the MR RF antenna device is provided to be proximal to a subject's perineum. By that, an MR signal with a high signal-to-noise ratio can be obtained while the patient bed is easy to use for an operator and the application of the MR RF antenna is comfortable for the subject. Moreover, a position in which the inner sides of the subject's legs are proximal to the side surfaces of the MR RF antenna device and the subject's perineum is proximal to the MR RF antenna device may inherently provide improved repeatability and reproducibility of a positioning of the MR RF antenna relative to the subject.

An even higher degree of repeatability and reproducibility of a subject's position can be obtained if the patient bed is furnished with fixation members arranged along each patient bed side, parallel to the patient bed lying direction, wherein each fixation member, in the at least one state of operation, is provided for being proximal at least to an outer side of one of the subject's legs.

In a preferred embodiment, the patient bed further comprises first guiding members arranged at each side of the patient bed, each of the first guiding members extending substantially perpendicular with regard to the lying direction, and first linking members attached to each of the fixation members that are provided to engage with the first guiding members to allow for an independent lateral guided movement of each of the fixation members along a corresponding one of the first guiding members, and a first locking device for locking each of the fixation members in at least one lateral position at the corresponding one of the first guiding member. This allows for fast patient fixation and a reduction or even an elimination of additional fixation devices. In principle, the first guiding members arranged at each side of the patient bed could also be formed integrally.

In another aspect of the present invention, the patient bed further comprises a second guiding member arranged at least along a portion of the patient bed and extending substantially parallel to the patient bed lying direction, at least one second linking member arranged at the MR RF antenna device that is provided to engage with the second guiding member to allow for a guided movement of the MR RF antenna device along the second guiding member, and a second locking device for locking the MR RF antenna device in at least one position at the second guiding member. By that, the MR RF antenna device can be adjusted to any position along the second guiding member, and a tight fit of the MR RF antenna device in close proximity to the patient perineum and a high signal-to-noise ratio can be achieved. The locking device may include a non-positive locking mechanism like a screw or a locking handle or any other mechanism that appears appropriate to the one of skills in the art.

In a further embodiment, the second guiding member of the patient bed has at least one recess and the second locking device is equipped with at least one fixing member that is provided to engage with the at least one recess in a positive locking. This allows for a fast, reliable, and repeatable positioning of the MR RF antenna device relative to the subject's perineum. In addition, the second guiding member may be furnished with an additional non-positive locking mechanism to lock the one fixing member in the position of positive locking with the at least one recess to enhance reliability of the positioning of the MR RF antenna device.

In yet a further embodiment, the patient bed may further comprise at least a second MR RF antenna device with at least one MR RF antenna that is enclosed in a housing, wherein the housing is integrally formed with one of the fixation members. By that, an MR signal with a high signal-to-noise ratio with an improved repeatability and reproducibility of a positioning of the MR RF antenna relative to the subject can be obtained for an upper part of the abdominal region, for instance for kidney imaging, while at the same time the patient bed is easy to use for an operator, the application of the MR RF antenna is comfortable for the subject, allowing for fast patient fixation with a reduction or even an elimination of additional fixation devices.

It is another object of the invention to provide a magnetic resonance (MR) radio frequency (RF) antenna device for use with a patient bed having an abdominal support portion for supporting an abdominal region of a subject, in particular during magnetic resonance-guided radiotherapy, and a lying direction intended for the subject to lie along, the MR RF antenna device comprising at least one MR RF antenna enclosed in a housing having two side surfaces opposing each other, wherein the MR RF antenna device is arrangeable at a top side of the patient bed in a patient bed center region. By that, an MR signal with a high signal-to-noise ratio can be obtained, while at the same time sanitizing requirements can be fulfilled in an easy and fast way, allowing for an efficient workflow to achieve a high patient throughput of therapy treatments.

Preferably and for achieving a large signal-to-noise ratio of an MR signal, the MR RF antenna of the MR RF antenna device is arranged at a position within a portion of the housing that encompasses a headmost one third of the housing relative to the lying direction of the patient bed.

In a preferred embodiment, the at last one MR RF antenna is provided for transmitting RF power during a first period of operation and for receiving magnetic resonance imaging signals at a second period of operation that is different from the first period of operation. Thereby, a compact design of the MR RF antenna can be achieved and RF interference by mutual coupling between separate transmitting and receiving MR RF antennae can be obviated.

In still another embodiment, the MR RF antenna device further comprises at least one magnetic resonance (MR) marker that is enclosed in the housing. The MR marker may allow for an easy and accurate subject and table positioning as well as for position verification.

In yet another embodiment, the MR RF antenna device further comprises at least one optical marker that is arranged at an outside of the housing. By that, the patient table can be positioned in a fast and reliable manner and a co-registration of an MR radiotherapy device coordinate system can be readily verified. This may be especially advantageous for MR based radiotherapy planning and simulation. Preferably, the at least one optical marker is placed at a top side of the MR RF antenna device that is distal to the patient bed in a state of operation.

In at least one sectional plane that is parallel to the top side of the patient bed the housing of the MR RF antenna device may have an oblong cross-sectional area that is elongated in the lying direction. Thereby, a natural lying position with a high degree of comfort for the subject can be achieved. Preferably, the housing is designed for an aspect ratio of the cross-sectional area of more than two, and most preferably, for an aspect ratio of more than three.

### BRIEF DESCRIPTION OF THE DRAWINGS

These and other aspects of the invention will be apparent from and elucidated with reference to the embodiments described hereinafter. Such embodiment does not necessarily represent the full scope of the invention, however, and reference is made therefore to the claims and herein for interpreting the scope of the invention.

In the drawings:

FIG. 1 illustrates a schematic top view of a patient bed in accordance with an embodiment of the invention,

FIG. 2 shows a schematic cross-sectional side view of the patient bed pursuant to the embodiment of FIG. 1 in a direction A-A′, and

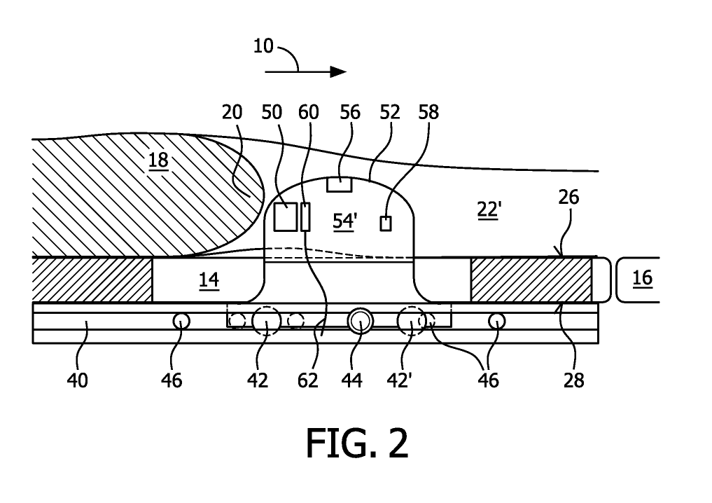

FIG. 3 shows a schematic cross-sectional side view of the patient bed pursuant to the embodiment of FIG. 1 in a direction B-B′.

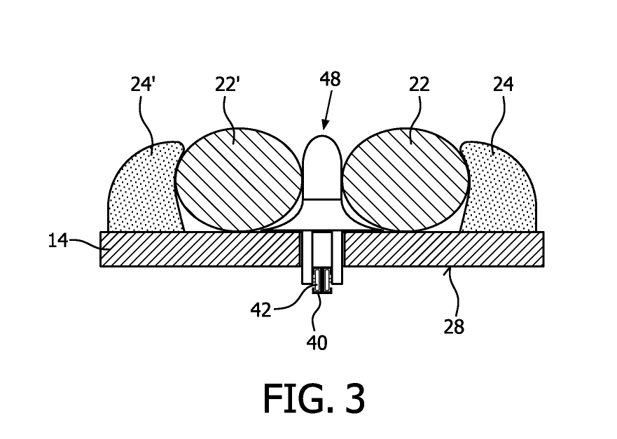

### DETAILED DESCRIPTION OF EMBODIMENTS

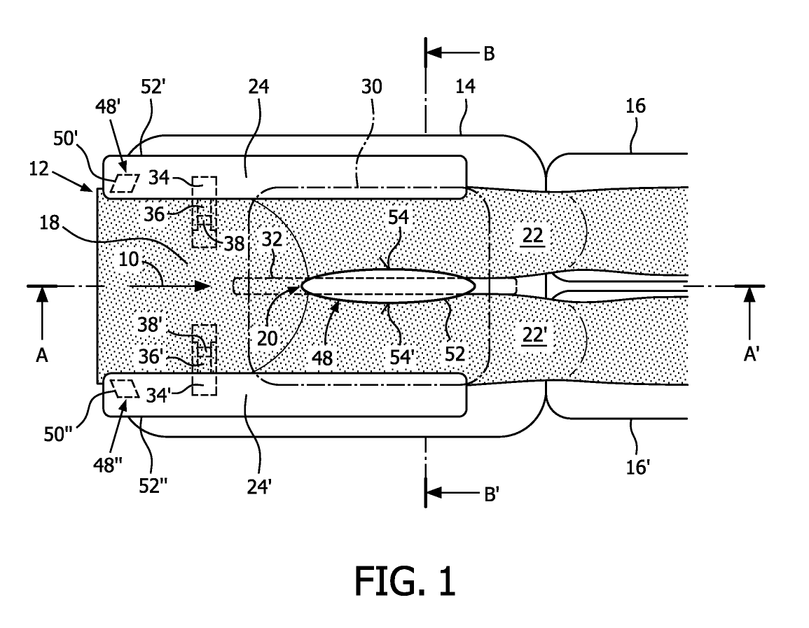

FIG. 1 illustrates a schematic top view of a patient bed in accordance with an embodiment of the invention. The patient bed is used in a radiotherapy (RT) system having a linear accelerator (LINAC) unit for accelerating elementary particles such as electrons or mesons for radiotherapy purposes. The RT system is guided by a magnetic resonance (MR) imaging device. Such an MR imaging-RT system is well known from prior art and is therefore not described in further detail herein. The use of the patient bed shall not be understood to be limited to MR imaging-RT systems, but shall rather encompass an application in MR imaging systems integrated with other therapy devices employing at least one out of ionizing radiation and ultrasound energy for therapy purposes, such as proton beam, ion beam, focused ultrasound (FUS) or shock wave devices.

The patient bed has an abdominal support portion 14 for supporting an abdominal region 16 of a subject 12 during magnetic resonance-guided radiotherapy and a lying direction 10 intended for the subject 12 to lie along. Further, the patient bed comprises two leg support portions 16 for supporting the subject's legs 22 from below, and two fixation members 24 designed as bars, one each being arranged along each patient bed side and parallel to the patient bed lying direction 10.

A position of the fixation members 24 lateral to the lying direction 10 can be adjusted. To this end, the patient bed is furnished with first guiding members 34 designed as ducts that are arranged at each side of the patient bed. Each of the first guiding members 34 extends substantially perpendicularly with respect to the lying direction 10. First linking members 36, each designed as a rail, are attached to each of the fixation members 24 at a face that is proximate to a patient bed top side 26 on that the subject is positioned and are provided to engage with the first guiding members 34 to allow for an independent lateral guided movement of each of the fixation members 24 along a corresponding one of the first guiding members 34. The first linking members 36 can engage with the first guiding members 34 by way of ball bearings or castors or any other means that appear to be appropriate to the one skilled in the art.

First locking devices 38 are provided for locking each of the fixation members 24 in any lateral position at the corresponding one of the first guiding members 34. The first locking devices 38 may be designed as mechanical brakes with brake pads that engage with the first guiding members 34 and that can be released by an operator pushing buttons that may be arranged at the fixation members 24. In principle, any other locking mechanism that the one skilled in the art considers to be appropriate can be applied.

Arranged at the top side 26 of the patient bed in a patient bed center region 30 is a magnetic resonance (MR) radio frequency (RF) antenna device 48 that comprises an MR RF antenna 50 of a coil type and a housing 52 having two side surfaces 54 opposing each other that extend substantially parallel to the lying direction 10. The center region is located relative to the patient bed's longitudinal axis, away from the longitudinal far ends of the patient table The MR RF antenna 50 is enclosed in the housing 52 such that the housing 52 completely encompasses the MR RF antenna 50. In a sectional plane that is parallel to the top side 26 of the patient bed, the housing 52 has an oblong cross-sectional area that is elongated along the lying direction 10, showing an aspect ratio larger than four. The MR RF antenna 50 is positioned inside the housing 52 within a housing portion that encompasses a headmost one third of the housing 52 relative to the lying direction 10 of the patient bed. The MR RF antenna 50 is provided for transmitting RF power during a first period of operation and for receiving magnetic resonance imaging signals at a second period of operation that is different from the first period of operation. The method of using a send/receive switch for feeding RF power to the MR RF antenna 50 at the first period of time and for feeding MR RF signals picked up by the MR RF antenna 50 to a signal processing unit is well known in the art and shall therefore not be described in detail herein.

The patient bed is equipped with a second guiding member 40 designed as a rail and arranged along the abdominal support portion 14 of the patient bed, extending substantially parallel to the patient bed lying direction 10 and parallel to a patient bed underside 28. Second linking members 42 designed as castors are arranged at the MR RF antenna device 48 that are provided to engage through an opening 32 in the patient bed with the second guiding member 40 to allow for a guided movement of the MR RF antenna device 48 along the second guiding member 40. The second guiding member 40 has a plurality of equidistantly spaced recesses designed as through openings in a direction perpendicular to the lying direction 10. A second locking device 44 is attached to the MR RF antenna device 48 that comprises a fixing member designed as a spring-loaded bolt (not shown) that is provided to engage in a locking position with one of the recesses building a positive locking. The positive locking can be released by pulling back the bolt with a handle (not shown) provided at an outside of the housing 52 of the MR RF antenna device 48 against spring force of the spring-loaded bolt.

While lying on the patient bed along the lying direction 10, the subject 12 is supported by the abdominal support portion 14 at the abdominal region 18 and at both legs 22 by the two leg support portions 16. Actually, the subject 12 is also supported by a backrest portion of the patient bed which for clarity purposes is not shown.

In preparation of a state of operation, the position of the MR RF antenna device 48 is adjusted with a released positive locking along the second guiding member 40 so that a portion of the MR RF antenna device 48 that is headmost with respect to the lying direction 10 is proximal to a subject's perineum 20. In this position, the positive locking is re-established by release of the handle.

The subject's legs 22 are then positioned so that each side surface 54 of the MR RF antenna device 48 is proximal to an inner side of each of the subject's legs 22. Then, the first locking devices 38 of the fixation members 24 are released by pushing the corresponding buttons, and the fixation members 24 are shifted along the first guiding members 40 toward the subject's leg 22 until in the state of operation, each of the fixation members 24 is proximal to an outer side of one of the subject's legs 22. In this position, the first locking devices 38 are re-engaged to lock the fixation members 24 in their position at the first guiding members 34. Thus, a well-defined relative position of the subject 12 and the MR RF antenna 50 can quickly and reproducibly be established in which the MR RF antenna 50 is located closely to the subject's perineum 20. At the same time, the subject's position is fixed by the fixation members 24 with a high degree of comfort for the subject 12, and further fixation means can be obviated.

A fast and reliable alignment of the patient bed with respect to a coordinate system of the MR imaging system can be conducted by employing an optical marker 56 that is arranged at a top side which is distal to the patient bed top side 26 in the state of operation, at an outside of the housing 52 of the MR RF antenna device 48.

In the state of operation, an MR marker 58 that is formed by an amount of a magnetic resonance-active species of nuclei enclosed in the housing 52 provides MR RF signals upon RF excitation that can be picked up by RF antennae of the MR imaging-guided RT system, thereby allowing for an easy and accurate subject and patient bed positioning as well as for position verification.

Furthermore enclosed in the housing 52 is a radiation detecting device 60. The radiation detecting device 60 is arranged at a position within the portion of the housing 52 that encompasses the headmost one third of the housing 52 relative to the lying direction 10 of the patient bed, so that, in the state of operation, the radiation detecting device 60 is located close to but outside regions of radiation exposure within the subject 12. An output cable 62 of the radiation detecting device 60 exits the housing 52 via a cable feed through to provide a signal corresponding to the radiation intensity to a control unit of the MR imaging-guided RT system (not shown). Alternatively, the signal corresponding to the radiation intensity could be transferred in a wireless way to the control unit. The one skilled in the art is familiar with devices and methods therefor. The control unit is provided to integrate the signal over time to determine a stray dose at the known location for quality assurance purposes, and for detection of deviations from pre-determined planning data that are stored in a memory of the control unit.

The patient bed has second MR RF antenna devices 48′, 48″ with MR RF antennae 50′, 50″ that are identical to the one in the first MR RF antenna device 48. The second MR RF antennae devices 48′, 48″ are each enclosed in a housing 52′, 52″ that is integrally formed with one of the fixation members 24, 24′, as indicated in FIG. 1 by dashed lines. The second MR RF antennae devices 48′, 48″ allow for easy subject fixation with an improved repeatability and reproducibility of a positioning of the MR RF antenna relative to the subject and a high signal-to-noise ratio for MR imaging of the subject's kidneys.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### REFERENCE SYMBOL LIST

10 lying direction
12 subject
14 abdominal support portion
16 leg support portion
18 abdominal region
20 perineum
22 leg
24 fixation member
26 patient bed top side
28 patient bed underside
30 patient bed center region
32 patient bed opening
34 first guiding member
36 first linking member
38 first locking device
40 second guiding member
42 second linking member
44 second locking device
46 recess
48 MR RF antenna device
50 MR RF antenna
52 housing
54 side surface
56 optical marker
58 MR marker
60 radiation detecting device
62 output cable

1. A patient bed, having an abdominal support portion for supporting an abdominal region of the human subject during magnetic resonance-guided therapy further comprising:
the magnetic resonance radio frequency antenna device arranged at a top side of the patient bed in a patient bed center region;
two leg support portions for supporting the subject's legs from below, wherein, in at least one state of operation, each side surface of the MR RF antenna device is provided to be proximal to an inner side of each of the subject's legs; and
wherein, in the at least one state of operation, the MR RF antenna device is provided to be proximal to a subject's perineum.

2. The patient bed as claimed in claim 1, and having a lying direction intended for the subject to lie along, further comprising
fixation members arranged along each patient bed side, parallel to the patient bed lying direction, wherein each fixation member, in the at least one state of operation, is provided for being proximal at least to an outer side of one of the subject's legs.

3. The patient bed as claimed in claim 2, further comprising
first guiding members arranged at each side of the patient bed, each of the first guiding members extending substantially perpendicularly with respect to the lying direction;
first linking members attached to each of the fixation members that are provided to engage with the first guiding members to allow for an independent lateral guided movement of each of the fixation members along a corresponding one of the first guiding members;
first locking devices for locking each of the fixation members in at least one lateral position at the corresponding one of the first guiding members.

4. The patient bed as claimed in 2, further comprising
a second guiding member arranged at least along a portion of the patient bed and extending substantially parallel to the patient bed lying direction
at least one second linking member arranged at the MR RF antenna device that is provided to engage with the second guiding member to allow for a guided movement of the MR RF antenna device along the second guiding member;
a second locking device for locking the MR RF antenna device in at least one position at the second guiding member.

5. The patient bed as claimed in claim 4, wherein the second guiding member has at least one recess and the second locking device is equipped with at least one fixing member that is provided to engage with the at least one recess in a positive locking.

6. The patient bed as claimed in claim 2, further comprising
at least a second magnetic resonance radio frequency antenna device with at least one MR RF antenna that is enclosed in the housing, wherein the housing is integrally formed with one of the fixation members.

7. A magnetic resonance radio frequency antenna device for use with a patient bed having an abdominal support portion for supporting an abdominal region of a human subject, in particular during magnetic resonance-guided radiotherapy, and a lying direction intended for the subject to lie in, the MR RF antenna device comprising
at least one MR RF antenna enclosed in a housing having two side surfaces opposing each other, wherein the MR RF antenna device is arrangeable at a top side of the patient bed in a patient bed center region and wherein the magnetic resonance radiofrequency antenna device further comprises a radiation detecting device that is enclosed in the housing.

8. The MR RF antenna device as claimed in claim 7, wherein the at last one MR RF antenna is provided for transmitting RF power during a first period of operation and for receiving magnetic resonance imaging signals at a second period of operation that is different from the first period of operation.

9. The MR RF antenna device as claimed in claim 7, further comprising at least one magnetic resonance marker that is enclosed in the housing.

10. The MR RF antenna device as claimed in claim 7, further comprising at least one optical marker that is arranged at an outside of the housing.

11. The MR RF antenna device as claimed in claim 7, wherein the housing has, in at least one sectional plane that is parallel to the top side of the patient bed, an oblong cross-sectional area that is elongated in the lying direction.

12. A therapy system employing at least one out of ionizing radiation and ultrasound energy for therapy purposes that is guided by a magnetic resonance imaging device, comprising a patient bed with at least one MR RF antenna device as claimed in claim 7.
